(19) United States
(12) Patent Application Publication
(10) Pub. No.: US 2026/0257015 A1
(43) Pub. Date: Sep. 3, 2026

(54) DRUG DELIVERY ARRANGEMENT COMPRISING A SKIN PINCHING MECHANISM

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/718,761

(22) PCT Filed: Dec. 13, 2022
(86) PCT No.: PCT/EP2022/085645

§ 371 Date: Jun. 11, 2024

(30) Foreign Application Priority Data
Dec. 15, 2021 (EP) 21315277.0

(51) Int. Cl. A61M 5/20; A61M 5/32; A61M 5/42; A61M 5/46

(52) U.S. Cl. CPC A61M 5/2033; A61M 5/3202; A61M 5/326; A61M 5/425; A61M 5/46; A61M 2005/2006; A61M 2005/206; A61M 2005/208; A61M 2202/0007; A61M 2205/58; A61M 2210/04

(57) ABSTRACT

A drug delivery device is described. The drug delivery device includes a housing, the housing being provided to receive a needle, a skin pinching mechanism having a first mechanism member and a second mechanism member. The first mechanism member and the second mechanism member are movable relative to one another between a first axial position and a second axial position. The second mechanism member is arranged to abut the patient's skin and comprises a skin pinching feature. The skin pinching mechanism is configured such that relative movement between the first mechanism member and the second mechanism member is converted into movement of the skin pinching feature so as to exert a force on the patient's skin to cause the skin to bulge in a proximal direction.

### CROSS REFERENCE TO RELATED APPLICATIONS

The present application is the national stage entry of International Patent Application No. PCT/EP2022/085645, filed on Dec. 13, 2022, and claims priority to Application No. EP 21315277.0, filed on Dec. 15, 2021, the disclosures of which are incorporated herein by reference.

### BACKGROUND

To both reduce the risk of injury to a patient when delivering a drug and to ensure effective delivery of the drug into the patient's body, the depth of penetration of the needle into the patient's body or tissue is important. Problems may arise with commercially available drug delivery devices if the drug delivery device is to be used for children. Children, in general, have less tissue available, which can be penetrated by a needle than grown-ups.

### SUMMARY

The present disclosure facilitates improvements associated with drug delivery devices, particularly with respect to patient safety or the injection depth.

The present disclosure relates to a drug delivery device. The drug delivery device comprises a housing, the housing being provided to receive a drug container and/or a needle. The needle is expediently configured to pierce a skin of a patient. Through the needle, a medicament may be administered to the patient, e.g. into the patient's tissue.

In one embodiment, the needle, e.g. when received in the housing, is axially fixed relative to the housing. Thus, axial movement of the needle relative to the housing may be prevented, for example in the distal direction and/or in the proximal direction. The needle may be received in the housing of the drug delivery device. The present disclosure may also apply to devices comprising or provided to retain a movable needle, which is configured to move relative to the housing, e.g. driven by a spring, for piercing the skin and/or for a drug delivery operation.

In one embodiment, the needle may be in or may be brought into fluid communication with an interior of the drug container. The needle may be integrated into the drug container. The drug, e.g. a liquid medicament, is expediently arranged in the interior of the container. The drug container may be a syringe, e.g. a syringe with a preinstalled needle, such as a staked needle. Alternatively, the drug container may be a cartridge, which may have to be brought into fluid communication with a separate needle unit, e.g. by piercing a cartridge septum with the needle of the needle unit.

In one embodiment, the drug delivery device comprises a skin pinching mechanism having a first mechanism member and a second mechanism member. The first mechanism member and the second mechanism member are movable relative to one another between a first axial position and a second axial position.

An axial position in this context is a position on the longitudinal axis of the drug delivery device. Hence, in the context of the present disclosure, the term “axis” or axial may refer to a longitudinal axis of the drug delivery device, e.g. the main longitudinal axis of the device and/or the housing. The term “radial” or “radial direction” in the present disclosure refers to the direction perpendicular to the longitudinal axis of the drug delivery device, wherein the term “radially inward” or “radial inward direction” means the direction pointing in the radial direction toward the longitudinal axis, and the term “radially outward” or “radial outward direction” means the direction pointing in the radial direction away from the longitudinal axis.

Accordingly, the first axial position and the second axial position are offset relative to each other along the longitudinal axis of the drug delivery device. The second mechanism member is arranged to abut or bear on the patient's skin, e.g. during operation of the drug delivery device, and comprises a skin pinching feature. The first mechanism member may move towards the skin during the movement from the first axial position to the second axial position.

In one embodiment, the drug delivery device comprises a needle shroud. The needle shroud comprises a needle shroud body. The second mechanism member comprises a support body. The support body may be a radially immovable or rigid member at the distal end of the drug delivery device. The support body may be the distal end of the needle shroud body. The support body may have a cylindrical shape. The skin pinching feature may extend distally from the support body or be arranged at a distal end of the support body.

The term “distal” or “distal end” designates that end of the drug delivery device or a component thereof which is or is to be arranged closest to a dispensing end of the drug delivery device. The term “proximal end” designates that end of the device or a component thereof which is or is to be arranged furthest away from the dispensing end of the device. The distal end and the proximal end are spaced apart from one another in the direction of an axis. The axis may be the longitudinal axis of the drug delivery device arrangement or elements thereof. A “distal direction” is therefore the direction pointing towards the patient's skin, while a “proximal direction” is the direction pointing away from the patient's skin. Thus, the proximal direction is the opposite direction of the distal direction.

In one embodiment, the skin pinching mechanism is configured such that relative movement between the first mechanism member and the second mechanism member from the first axial position to the second axial position is converted into movement of the skin pinching feature so as to exert a force on the patient's skin to cause the skin to bulge in the proximal direction.

For moving the skin pinching feature, the first mechanism member may be arranged to abut the skin pinching feature in order to displace the skin pinching feature. This movement of the skin pinching feature may be used to manipulate the skin, e.g. to pinch the skin. The skin pinching feature may be configured to exert a force on or transfer a force to the patient's skin. The force may cause the skin to bulge in the proximal direction. The force may cause the patient's skin to bulge towards a tip of the needle. The force may cause the patient's skin to bulge towards a needle passage opening of the drug delivery device. The force which moves the skin pinching feature, preferably is patient exerted force, e.g. manually exerted force.

The skin pinching mechanism allows the penetration region of the skin to be lifted proximally and/or towards the needle. In this way, the thickness of the tissue available for needle penetration is increased and/or the skin may be adjusted to the penetration depth of the needle. Thus, the drug delivery device reduces the risk of injuring or causing discomfort to the patient when delivering the drug and/or ensures reliable delivery of the drug, especially when the patient is a patient with thinner tissue, such as a child. Adjustment of the skin area into which the needle enters can be achieved by pinching the skin in a way that causes the skin to move towards the needle. In this way, the tissue thickness is increased in the area where the needle penetrates or will penetrate the skin.

In one embodiment, the first mechanism member is the housing or at least a part of the housing. The housing may be the portion of the drug delivery device on which the drug delivery device is held by the patient. Accordingly, the movement of the first mechanism member relative to the second mechanism member may be effected by the patient without having to change the holding position of his hand and fingers thereof. This facilitates the handling of the drug delivery device considerably.

In one embodiment, the needle is axially locked to prevent movement relative to the first mechanism member when the first mechanism member is moved relative to the second mechanism member from the first axial position to the second axial position. This means that no axial relative movement between the needle and the first mechanism member is possible, e.g. in the proximal direction and/or in the distal direction. The needle can be inserted into the patient's skin when the first mechanism member is moved relative to the second mechanism member from the first axial position to the second axial position. Thus, the movement of the first mechanism member relative to the second mechanism member can occur together with the insertion of the needle into the skin.

In one embodiment, the needle is axially movable relative to the first mechanism member when the first and second mechanism members are in the second axial position. Thus, the insertion of the needle into the patient's skin can also occur after the first mechanism member has moved relative to the second mechanism member, e.g. by way of an automated needle insertion mechanism. This ensures that the needle does not pierce the patient's skin until the patient's skin has thickened due to pinching in the skin piercing area. This leads to an increased application safety of the drug delivery device.

In one embodiment, the skin pinching feature is movable relative to the support body between a first feature position and a second feature position. The skin pinching mechanism is configured such that when moved from the first feature position to the second feature position, the skin pinching feature changes its radial or rotational position relative to the support body to pinch the patient's skin. The first feature position may be the position where the skin pinching feature is farthest in the radial direction from the support body, wherein the second feature position may be the position where the skin pinching feature is closest to the support body in the radial direction. In order to change its radial position, the skin pinching feature may be pivotally connected to the support body. Apart from that, in order to change its rotational position, the skin pinching feature may be rotatably connected to the support body.

In one embodiment, the skin pinching feature may be connected to the support body by a hinge.

In one embodiment, the skin pinching feature is at least partially elastically deflectable. The material of the skin pinching feature may be elastic at least in the area where the skin pinching feature is connected to the support body. In the present context elasticity is the property of a body or material to change its shape when a force is applied and to return to its original shape when the applied force is removed. By being at least partially elastically deflectable, the skin pinching feature can be pivotally connected to the support body. In this case, the skin pinching feature and the support body may be formed more easily in one integral part.

In one embodiment, the skin pinching feature comprises a first portion and a second portion, wherein the first portion is connected to the support body and the second portion is disposed further from the support body than the first portion, and wherein the second portion comprises a skin contacting portion. The first portion and the second portion may be oriented in different directions. The skin pinching feature may be an arm. The arm may have a free end. The arm may be a bent arm. The arm may have a hook shape, e.g. in its distal end region. A tip of the hook, which may define the second portion of the arm and/or comprise the free end of the arm, may protrude laterally from the first portion of the arm. The tip of the hook may extend in a radial direction with respect to the longitudinal axis of the drug delivery device and thus inward. The second portion of the skin pinching feature may define a bearing surface, with which the skin pinching feature bears on the skin. The hook may be the second portion, wherein the hook tip may define the skin contact portion. The area that connects the hook to the support body can be the first portion. This assists in manipulating the patient's skin by pinching the skin. The distance in the radial direction between the second portion and the support body may be greater in the first feature position than in the second feature position.

In one embodiment, the skin pinching mechanism is configured such that when the first and second mechanism members are in the second axial position, the skin pinching feature is biased to the position that the skin pinching feature is in when the first and second mechanism members are in the first axial position. The skin pinching feature may be configured to be held in the first feature position by a spring force acting on the first and/or second portions when no additional force is applied to the first or second portions. When the first mechanism member is moved relative to the second mechanism member from the first axial position to the second axial position in order to pinch the skin, the spring force must be overcome to move the skin pinching feature from the first feature position to the second feature position.

In one embodiment, the skin pinching feature comprises a rotatable body. The rotatable body may be rotatably mounted on the support body. By being rotatably mounted on the support body, the skin pinching feature can be rotatably connected relative to the support body. The rotatable body may be located at the distal end of the support body. The rotatable body may include a skin contact surface configured such that the skin contact surface extends angularly about the axis of rotation of the rotatable body. The material of the surface of the skin contact surface may be such that when the skin contact surface is placed on the skin of the patient, the friction between the skin contact surface and the skin of the patient prevents relative movement of the patient's skin with respect to the skin contact surface. Accordingly, an inward rotational movement of the rotatable body causes the skin to be displaced, pinched and thus bulged in the proximal direction.

In one embodiment, the second mechanism member comprises at least two skin pinching features. The skin pinching features may be equidistantly arranged along a circumference of the support body. The skin pinching features can be evenly distributed in the circumferential direction of the support body. The skin pinching features can be diametrically opposed in the circumferential direction of the support body. The skin pinching features can be movable relative to each other, e.g. in opposite rotational directions or radially inwardly towards one another.

In one embodiment, the second mechanism member comprises a plurality of skin pinching features that are arranged directly adjacent to each other. The support body may be the distal end of the needle shroud body. The needle shroud may be provided to cover the needle. The needle shroud may be provided to cover the needle before the needle pierces the skin and/or after the needle has been removed from the skin, e.g. after completion of the drug delivery operation. Before the drug delivery operation is commenced, the needle shroud may protrude distally from the housing, e.g. to cover the tip of the needle (such as by axially extending beyond the tip of needle in the distal direction). For the drug delivery operation, the needle shroud may be displaced proximally relative to the housing. After completion of the drug delivery operation, the needle shroud may be moved distally relative to the housing, e.g. to cover the tip of needle.

In one embodiment, the skin pinching mechanism comprises an interaction feature. The interaction feature may be configured to lock the skin pinching feature against radial or rotational movement relative to the support body when the first and second mechanism members are in the second axial position. The interaction feature may be configured to lock the skin pinching feature against radial or rotational movement relative to the support body when the skin pinching feature is in the second feature position. The interaction feature may be attached to or be part of the first mechanism member. Accordingly, the interaction feature may be attached to the housing. Alternatively, the interaction feature may be attached to or be part of the second mechanism member. The interaction feature may be attached to or be part of the skin pinching feature.

In one embodiment, the interaction feature is located on the inner surface of the housing.

In one embodiment, the interaction feature is located in the distal end region of the housing. The interaction feature may point or be radially oriented inward.

In one embodiment, the interaction feature can be a protrusion oriented radially with respect to the longitudinal axis of the drug delivery device.

In one embodiment, the rotatable body comprises a recess. The recess may be configured to engage the interaction feature. The recess may have a complementary shape to the interaction feature. The recesses may be configured such that when the first and second mechanism members are moved from the first to the second axial position, the interaction feature is pushed into the recess such that the recess is rotated from the first feature position to the second feature position to pinch the patient's skin.

In one embodiment, the drug delivery device comprises a shroud spring. The shroud spring may be operatively coupleable to or coupled to the needle shroud in order to move the needle shroud, e.g. into the distal direction relative to the housing. The force of the shroud spring may have to be overcome in order to move the needle shroud in the proximal direction. In a final position, e.g. after the drug delivery operation has been completed and the device has been removed from the skin, the needle shroud may be locked against proximal movement with respect to the housing, such as by a locking mechanism.

In one embodiment, the needle shroud is an activation member. The activation member is a member which may have to be moved relative to the housing in order to enable triggering of the drug delivery operation or to trigger the drug delivery operation. Enable triggering of the drug delivery operation may comprise that in addition to movement of the activation member another member such as a trigger member has to be actuated, e.g. a trigger button has to be pressed.

In one embodiment, the drug delivery device further comprises an energy storage unit. The energy storage unit may be a drive spring or another type of energy source such as a gas reservoir. The drug delivery device is configured to perform a drug delivery operation, e.g. using energy obtainable from the energy storage unit. The energy storage unit may be configured to provide energy for the drug delivery operation of the drug delivery device. The energy may be used to drive a drive member, e.g. a plunger rod, of the drug delivery device in order to dispense drug from the drug container. For the drug delivery operation, the drive member may be displaced in the distal direction relative to the housing by the energy provided by the energy storage unit.

In one embodiment, the drug delivery device is an autoinjector. In an autoinjector the energy for the drug delivery operation may be prestored in the energy storage unit. That is to say, the patient does not have to provide the energy for the drug delivery operation, e.g. when preparing the device for use. Rather, this energy may be preloaded into the system by the manufacturer. For example, a drive spring may be pre-stressed or pre-biased to provide the energy for the drug delivery operation.

In one embodiment, the drug delivery device comprises a plunger release mechanism configured to have a first state and a second state, wherein the stored energy of the energy storage unit is released when the plunger release mechanism is in the second state.

In one embodiment, the plunger release mechanism is switched from the first state to the second state after the needle is moved relative to the second mechanism member.

In one embodiment, the needle is configured such that after the energy storage unit has released the energy, the needle moves in a proximal direction relative to the second mechanism feature.

We note that features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

### BRIEF DESCRIPTION OF THE FIGURES

FIG. 1 illustrates a schematic view of a first or a second embodiment of the drug delivery device according to the present disclosure.

FIG. 2A illustrates a sectional side view of a portion of the first embodiment prior to a dispensing operation.

FIG. 2B illustrates another sectional side view of the portion of the first embodiment during a dispensing operation.

FIG. 3A illustrates a sectional side view of a portion of the second embodiment prior to a dispensing operation.

FIG. 3B illustrates another sectional side view of the portion of the second embodiment during a dispensing operation.

FIG. 4A illustrates a sectional side view of a portion of a third embodiment prior to a dispensing operation.

FIG. 4B illustrates another sectional side view of the portion of the third embodiment during a dispensing operation.

FIG. 5 illustrates a top view of a distal end of a drug delivery device according to the first or second embodiment.

FIG. 6 illustrates a top view of a distal end of a drug delivery device according to a further embodiment of the drug delivery device.

FIG. 7 illustrates a top view of a distal end of a drug delivery device according to yet another embodiment of the drug delivery device.

### DETAILED DESCRIPTION

Identical elements, elements of the same kind and identically or similarly acting elements may be provided with the same reference numerals in the drawings.

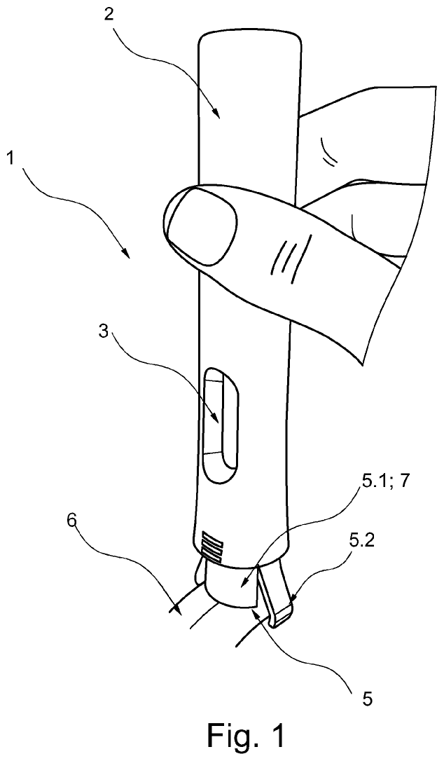

The embodiments depicted in FIGS. 1 to 7 relate to a drug delivery device 1 that is very similar to the device disclosed in WO 2015/004052 A1, the entire disclosure content of which is incorporated herein by reference for all purposes, especially with respect to the design of the drive mechanism or “plunger release mechanism” as it is termed therein.

The drug delivery device 1 having a housing 2, a drug container 3 and a needle 4 (needle 4 not shown in FIG. 1). The housing 2 being provided to receive the drug container 3, wherein the drug container 3 in FIG. 1 is visible through an opening or window in the housing 2. The housing 2 expediently covers the majority of the length of the drug delivery device 1, e.g. 60% or more or 70% or more or 80% or more of the entire length of the drug delivery device 1.

The housing 2 is provided to retain and/or retains the drug container 3. Medicament, e.g. liquid medicament, is arranged in the drug container 3. The housing 2 is provided to retain and/or retains the needle 4. In other words, a needle 4 may be arranged or arrangeable in the housing 2. The needle 4 can be an integral part of the drug container 3, e.g. (permanently or releasably) connected to a drug container body, or separate from the drug container. In the first case, the drug container 3 may be a syringe. In the second case, the drug container 3 may be a cartridge. In case a cartridge is used as drug container 3, initially, the drug container and the needle 4 can be fluidly disconnected and fluid communication between the drug container interior and the needle is only established during operation of the drug delivery device 2.

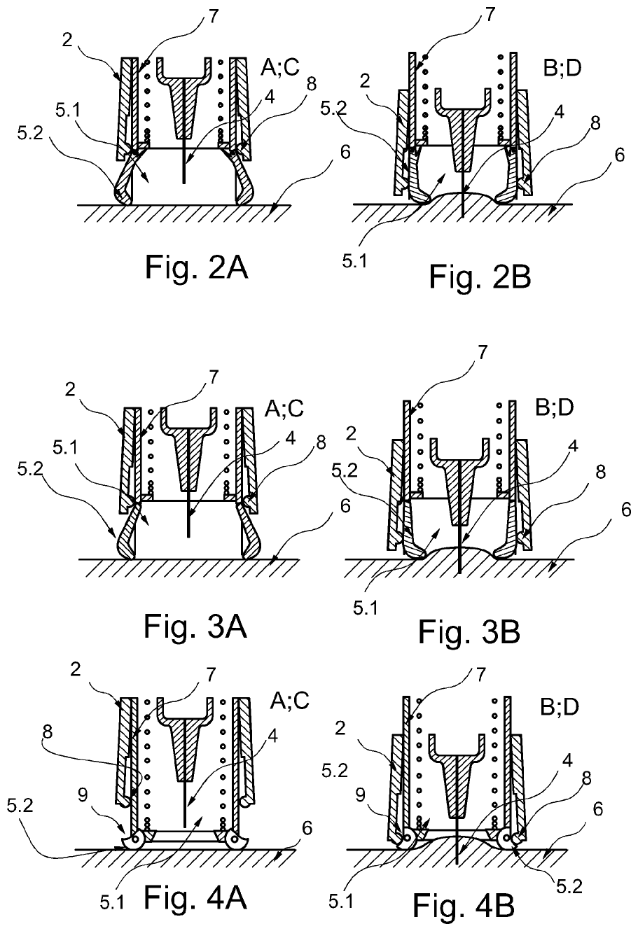

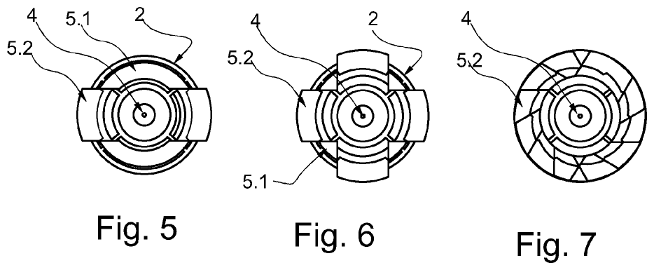

As depicted in FIG. 1, the drug delivery device 1 comprises skin pinching mechanism. The skin pinching mechanism has a first mechanism member and a second mechanism member 5. In the shown embodiments of FIGS. 1 to 4B the first mechanism member is the housing 2, wherein the second mechanism member 5 comprises a support body 5.1 and a skin pinching feature 5.2. The housing 2 is axially movable relative to the support body 5.1 and the skin pinching feature 5.2. The embodiments shown have two skin pinching features 5.2 that are diametrically opposed to each other. However, as seen in FIGS. 5, 6, and 7, all of the following embodiments depicted in FIGS. 2A to 4B may have more than two skin pinching features 5.2. As shown in FIG. 7, the skin pinching features 5.2 can also be arranged adjacent to each other. Similarly, the embodiments depicted in FIGS. 2A to 4B may have only a single skin pinching feature 5.2.

As depicted in FIGS. 2A to 4B, the first mechanism member, i.e. the housing 2, and the second mechanism 5 member are movable relative to one another between a first axial position A (FIGS. 2A, 3A, 4A) and a second axial position B (FIGS. 2B, 3B, 4B). In addition, the skin pinching features 5.2 are movable relative to the support body 5.1 between a first feature position C (FIGS. 2A, 3A, 4A) and a second feature position D (FIGS. 2B, 3B, 4B) relative to the housing. The relative movement between the first mechanism member and the second mechanism member 5 is converted into radial (FIG. 2A to 3B) or rotatable (FIGS. 4A, 4B) movement of the skin pinching feature 5.2 towards the longitudinal axis. This movement is used to pinch the skin 6 of a patient in order to insert the needle 4 into the pinched area.

As depicted in FIGS. 2A to 4B, the needle 4 is not movable in the axial direction relative to the housing 2. According to this, the needle 4 moves together with the housing 2 when the housing is moved relative to the skin pinching mechanism in an axial direction. It should be noted, however, that in a further embodiment not shown, the needle may also be movable relative to the housing. In this embodiment, it is possible that the needle is moved in the distal direction only after positions B and D have been reached, and is thus inserted into the skin.

The two skin pinching features 5.2 according to the embodiments depicted in FIG. 2A to 3B are designed as pivot-mounted (FIGS. 2A, 2B) or elastically deflectable arms (FIGS. 3A, 3B) provided at the support body 5.1. At the distal end region of the arms, the arms are curved to form inward-facing hooks. The hook shape, together with the friction, helps the skin 6 to be gripped well by the arms.

The support body 5.1 according to the embodiments depicted in FIG. 2A to 4B is not radially movable relative to the housing 2. In the embodiments shown in FIG. 1 to 4B, the support body 5.1 is the distal end portion of a needle shroud 7, e.g. a generally cylindrical portion. The support body 5.1 and the skin pinching features 5.2 are not movable in axial direction relative to each other. The needle shroud 7 protrudes distally from the housing 2. The needle shroud 7 is movable in a proximal direction relative to the housing 2. The needle shroud 7 may also be movable in a distal direction relative to the housing 2.

Movement of the two skin pinching features 5.2 relative to each other and relative to the support body 5.1 is brought about by the patient pressing the housing 2 distally onto the patient's skin 6. This causes the housing 2 to move in the axial direction relative to the skin pinching features 5.2 and the needle shroud 7. The inner diameter of the housing 2 is dimensioned so that the needle shroud 7 can move within it in the axial direction. The skin pinching features 5.2 can change the dimension of the area which they surround in radial direction since the skin pinching features 5.2 are movable relative to the support body 5.1. The maximum width in radial direction in which the skin pinching features 5.2 are able to extend is larger than the inner diameter of the housing 2. The skin pinching features 5.2 are held in the position where the skin pinching features have their maximum dimensions in radial direction by a spring force. The spring force can be provided either by an external spring element, as seen in FIGS. 2A and 2B, or by an elastic material composition in the area where the skin pinching feature 5.2 is connected to the support body 5.1, as seen in FIGS. 3A and 3B. Therefore, in order to be able to slide the housing 2 over the skin pinching features 5.2, the spring force which holds the skin pinching features 5.2 in the first feature position C must be overcome. If this is done, the housing 2 can be pushed over the skin pinching features 5.2, wherein the skin pinching features are moved towards each other against the spring force. If the skin pinching features 5.2 are in contact with the patient's skin during this movement, the distal movement of the housing 2 causes the skin 6 that lies between the two skin pinching features 5.2 and the needle shroud 7, or support body 5.1, to pinch and bulge in the proximal direction.

The drug delivery device 1 according to the embodiments of FIGS. 1 to 7 comprises an energy storage unit, e.g. a drive spring, such as a compression spring, (not shown). The energy storage unit is arranged to drive a plunger rod in a distal direction relative to the drug container 3 during the drug delivery operation. During this movement, a stopper, which is movably retained in the drug container 3 and may seal the drug container proximally, can be displaced towards an outlet of the drug container to dispense the drug or medicament retained within the drug container 3 through the outlet. The outlet may be formed or defined by the needle 4. Other potential drive energy sources different from a spring comprise an electrical power cell or battery for driving the plunger rod by a motor or a reservoir suitable to provide gas pressure, where the gas pressure can be used to drive the drug delivery operation.

The drug delivery device 1 is an autoinjector. The energy for driving the drug delivery operation in an autoinjector may be provided by components integral to the drug delivery device 1 and does not have to be loaded into the device by the patient during the operation of the device as is the case in many spring driven pen-type variable dose injectors, where, usually, the energy is loaded into the spring by the patient during a dose setting procedure.

The drug delivery device 1 expediently is a single shot device, i.e. it is provided to dispense only one dose. The drug delivery device 1 may be disposable drug delivery device, that is to say a device which is disposed of after its use. The device may be a pen-type device. The medicament container 3 and/or the needle 4 can be axially secured within the drug delivery device 1, e.g. within the housing 2, or can be movable relative to the housing 2, e.g. for piercing the skin. In the first case, the patient may have to perform the movement for piercing the skin 6 with the needle 4. In the second case, piercing of the needle 4 may be driven by a needle insertion mechanism of the drug delivery device 1.

Furthermore the drug delivery device 1 comprises a plunger release mechanism (not shown) configured to have a first state and a second state. In the embodiments shown, the plunger release mechanism prevents the release of energy from the energy storage unit in the first state and allows the release of energy from the energy storage unit in the second state. In FIGS. 2A, 3A and 4A, the plunger release mechanism is in the first state. The plunger release mechanism may be a mechanical lock which prevents movement of the plunger rod in the distal direction.

As shown in FIGS. 2A through 4B, the operation of the skin pinching mechanism is assisted by two interaction features 8 disposed on the inner peripheral surface of the housing 2 in the distal end region of the housing 2. These interaction features 8 are configured as protrusions that directly abut the outer surface of the second mechanism member 5. When the housing 2 is moved in a distal direction relative to the second mechanism member 5, the interaction features 8 slide along the outer surface of the skin pinching features 5.2. Since the housing 2 is radially immobile, the skin pinching features 5.2 are forced radially inward by the interaction features 8. Thus, the interaction features 8 are configured to transfer the skin pinching features 5.2 from the first feature position C to the second feature position D.

As can be seen in FIGS. 4A and 4B, the skin pinching features 5.2 may have a corresponding shape to the interaction features 8. In the embodiment shown in FIGS. 4A and 4B, the skin pinching features 5.2 are shaped as rotatable bodies. The rotatable bodies are located at the distal end of the support body 5.1. The rotatable bodies include a skin contact surface configured such that the skin contact surface extends angularly about the axis of rotation of the rotatable bodies. The rotatable bodies have recesses 9 corresponding to the shape of the interaction features 8. The recesses 9 face away from the needle exit opening, i.e. from the area enclosed by the skin pinching feature 5.2 and the support body 5.1, in the radial direction. When the housing 2 is moved relative to the needle shroud 7 in the distal direction from the first axial position A to the second axial position B, the interaction features 8 abut the recesses 9 of the rotatable bodies. Since the rotatable bodies are rotatably mounted, distal movement of the housing 2 thus causes the rotatable bodies to rotate inwardly into the region enclosed by the skin pinching features 5.2 and the support body 5.1. The friction of the rotatable bodies on the skin 6 causes the skin between the rotatable bodies to pinch together and move in a proximal direction.

Although not explicitly shown, the skin pinching features 5.2 of the embodiments depicted in FIGS. 2A to 3B may also have corresponding recesses 9 that can interact with the interaction features 8 of the housing 2. For example, the recesses 9 can be located at the back of the arms, i.e. at the side of the arms that are inclined away from the longitudinal axis of the drug delivery device 1 in the radial direction.

The second axial position B and/or the second feature position D may be a trigger position. When the first mechanism member, i.e. the housing 2, and the second mechanism member 5 are in the first axial position A and the skin pinching features 5.2 are in the first feature position C, the drug delivery operation can be initialized by moving the housing 2 together with the needle 4 relative to the needle shroud 7. This movement advances the needle 4 in the distal direction so that the needle 4 pierces the skin 6. In addition, this movement causes the locking of the plunger release mechanism to be released. The plunger release mechanism releases the energy of the energy storage unit so that the drug is injected into the bulged area of the skin 6, which is pinched by the skin pinching features 5.2. After the drug delivery operation has been completed, the drug delivery device 1 may be removed from the skin 6. The needle shroud 7 may be biased relative to the housing 2. Thus, when the skin pinching features 5.2 and the support body 5.1 are removed from the skin 6, the needle shroud 7 is moved distally beyond the first axial position A into a final locked position relative to the housing 2. In this position the needle shroud 7 is expediently axially locked relative to the housing 2 against movement in the proximal direction, e.g. by a locking engagement between a locking feature of the shroud 7 and the housing 2. As it is axially locked, the needle shroud 7 can no longer be displaced proximally relative to the housing 2. This protects the patient from needle stick injuries after use.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a patient prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the patient). Another multi-dose container system may involve a needle-based injection device with an integrated non-replaceable container. In such a system, each container holds multiple doses, the size of which may be fixed or variable (pre-set by the patient).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### REFERENCE NUMERALS

drug delivery device 1
housing 2
drug container 3
needle 4
second mechanism member 5
support body 5.1
skin pinching feature 5.2
skin 6
needle shroud 7
interaction feature 8
recess 9
first axial position A
second axial position B
first feature position C
second feature position D

1.-21. (canceled)

22. A drug delivery device for dispensing a drug to a patient, the drug delivery device comprising:
a housing configured to receive a needle; and
a skin pinching mechanism having a first mechanism member and a second mechanism member,
wherein the first mechanism member and the second mechanism member are movable relative to one another between a first axial position and a second axial position,
wherein the second mechanism member is arranged to abut skin of the patient and comprises a skin pinching feature, and
wherein the skin pinching mechanism is configured such that relative movement between the first mechanism member and the second mechanism member from the first axial position to the second axial position is converted into movement of the skin pinching feature, the movement of the skin pinching feature exerting a force on the skin of the patient to cause the skin to bulge in a proximal direction.

23. The drug delivery device according to claim 22,
wherein the first mechanism member is the housing.

24. The drug delivery device according to claim 22,
wherein the skin pinching feature is at least partially elastically deflectable.

25. The drug delivery device according to claim 22,
wherein the second mechanism member comprises a support body, wherein the skin pinching feature is movable relative to the support body between a first feature position and a second feature position, and
wherein the skin pinching mechanism is configured such that when moved from the first feature position to the second feature position, the skin pinching feature changes its radial or rotational position relative to the support body to pinch the skin of the patient.

26. The drug delivery device according to claim 25,
wherein the skin pinching feature is rotatably or pivotally connected to the support body.

27. The drug delivery device according to claim 25,
wherein the skin pinching mechanism is configured such that when the first and second mechanism members are in the second axial position, the skin pinching feature is biased to a position that the skin pinching feature is in when the first and second mechanism members are in the first axial position.

28. The drug delivery device according to claim 25, wherein
the skin pinching feature and the support body are formed in one integral part.

29. The drug delivery device according to claim 25,
wherein the skin pinching feature comprises a first portion and a second portion, wherein the first portion is connected to the support body and the second portion is disposed further away from the support body than the first portion, and wherein the second portion comprises a skin contacting portion.

30. The drug delivery device according to claim 25,
wherein the skin pinching feature comprises a rotatable body, and wherein the rotatable body is rotatably mounted on the support body.

31. The drug delivery device according to claim 22,
wherein the second mechanism member comprises at least two skin pinching features, and wherein the skin pinching features are arranged equidistantly in a circumferential direction of the support body.

32. The drug delivery device according to claim 22,
wherein the drug delivery device comprises a needle shroud, and wherein the support body is the needle shroud.

33. The drug delivery device according to claim 22,
wherein the skin pinching mechanism comprises an interaction feature, and
wherein the interaction feature is configured to lock the skin pinching feature against radial or rotational movement relative to the support body when the first and second mechanism members are in the second axial position.

34. The drug delivery device according to claim 22,
wherein the needle is axially locked to prevent movement relative to the first mechanism member when the first mechanism member is moved relative to the second mechanism member from the first axial position to the second axial position.

35. The drug delivery device according to claim 22,
wherein the needle is axially movable relative to the first mechanism member when the first and the second mechanism members are in the second axial position.

36. The drug delivery device according to claim 22, wherein
the drug delivery device is an autoinjector.

37. The drug delivery device according to claim 22, wherein the housing is provided to retain and/or retains a drug container and wherein a medicament is arranged in the drug container.

38. The drug delivery device according to claim 22, wherein the drug delivery device comprises a needle shroud and wherein the inner diameter of the housing is dimensioned so that the needle shroud can move within it in axial direction.

39. The drug delivery device according to claim 22, wherein the second mechanism member comprises at least two skin pinching features.

40. The drug delivery device according to claim 39, wherein the housing can be pushed over the skin pinching features, wherein the skin pinching features are moved towards each other.

41. A method comprising:
dispensing a medicament with the drug delivery device, wherein the drug delivery device comprises:
a housing configured to receive a needle; and
a skin pinching mechanism having a first mechanism member and a second mechanism member,
wherein the first mechanism member and the second mechanism member are movable relative to one another between a first axial position and a second axial position,
wherein the second mechanism member is arranged to abut the skin of the patient and comprises a skin pinching feature, and
wherein the skin pinching mechanism is configured such that relative movement between the first mechanism member and the second mechanism member from the first axial position to the second axial position is converted into movement of the skin pinching feature, the movement of the skin pinching feature exerting a force on the skin of the patient to cause the skin to bulge in a proximal direction.
